(19) United States
(12) Patent Application Publication
(10) Pub. No.: US 2025/0318774 A1
(43) Pub. Date: Oct. 16, 2025

(54) BIOIMPEDANCE ANALYSIS FOR TISSUE ASSESSMENT

(71) Applicant: Grand Valley State University, Allendale, MI (US)

(72) Inventors: Ross Sherman, Grand Rapids, MI (US); Brian Hatzel, Grand Haven, MI (US)

(73) Assignee: Grand Valley State University, Allendale, MI (US)

(21) Appl. No.: 19/171,492

(22) Filed: Apr. 7, 2025

Related U.S. Application Data
(60) Provisional application No. 63/632,665, filed on Apr. 11, 2024.

(51) Int. Cl. A61B 5/00; A61B 5/053; G16H 10/60; G16H 50/70

(52) U.S. Cl. CPC A61B 5/45; A61B 5/053; A61B 5/4842; A61B 5/7475; G16H 10/60; G16H 50/70

(57) ABSTRACT

An assessment method for identifying tissue conditions and injuries includes supplying an excitation signal via a plurality of source electrodes into a plurality of anatomical segments of a patient and detecting characteristic signals resulting from a first impedance of a first segment. The first impedance is compared to baseline impedance or a bilateral impedance of a symmetric portion of the patient. Based on the comparison, a tissue condition may be assessed that may correspond to an injury or a response to overloading.

### CROSS-REFERENCE TO RELATED APPLICATIONS

This application claims priority under 35 U.S.C. § 119 (e) and the benefit of U.S. Provisional Application No. 63/632,665 entitled BIOIMPEDENCE ANALYSIS FOR TISSUE ASSESSMENT, filed on Apr. 11, 2024, by Sherman et al., the entire disclosure of which is incorporated herein by reference.

### BACKGROUND OF THE INVENTION

The disclosure relates to methods for injury and recovery assessment based on bioimpedance analysis. In various areas of medicine, the assessment of the presence and severity of injuries is based on subjective measurements derived from examination with limited objective information. For example, examination techniques may vary widely among healthcare practitioners, and patients may report disability or discomfort based on highly subjective pain measures. In general, the subjective nature of a patient assessment may be improved with objective information providing indications of the conditions of the corresponding tissue. The following disclosure provides for improved methods and detection equipment that may provide for a preliminary and serial assessment of tissue condition and serve as an adjunct to inform a diagnosis and effective treatment of various patient conditions including tissue assessment, injury severity assessment, tissue characteristics, tissue recovery, and/or identifying the presence of tissue abnormalities.

### SUMMARY OF THE INVENTION

In various implementations, the disclosure provides for methods of applying bioimpedance analysis and equipment to assess musculoskeletal injury. In operation, the method may compare the impedance of bilaterally symmetric segments of patient anatomy to detect the existence and severity of one or more injuries or abnormalities based on a comparative analysis. In operation, the assessment method may begin by conductively connecting a plurality of source electrodes and sense electrodes across a first anatomical segment and a second anatomical segment of a patient. The anatomical segments may correspond to portions of a patient anatomy that may be mirrored laterally across a sagittal plane of the patient. In this way, the assessment method may compare the electrical response of the anatomical segments of the patient to identify abnormalities or tissue responses that may be asymmetric in impedance response.

In operation, the electrical response of the anatomical segments of the patient may be measured by applying an excitation signal to the source electrodes and detecting resulting characteristic signals representative of the bioimpedance across each of the anatomical segments. Based on differences in the first impedance and the second impedance associated with the corresponding symmetric segments of the patient anatomy, the assessment method may be implemented to identify the existence and the relative severity of an injury associated with the anatomical segments. In this way, the assessment method may provide for supplemental guidance that may improve the detection of injuries or compromised tissue conditions for follow up by diagnostic methods (MRI, CT, Xray), physician assessment, and/or therapeutic intervention such as physical therapy, and/or surgical procedures.

These and other features, objects and advantages of the present invention will become apparent upon reading the following description thereof together with reference to the accompanying drawings.

### BRIEF DESCRIPTION OF THE DRAWINGS

FIG. 1 is an illustrative process diagram demonstrating an assessment method;

FIG. 2A is a schematic diagram demonstrating an octopolar segmental bioelectrical impedance analysis model;

FIG. 2B is a schematic diagram demonstrating a direct tetrapolar segmental bioelectrical impedance analysis model;

FIG. 3 is a block diagram demonstrating an exemplary bioelectrical impedance analyzer;

FIG. 4 is a pictorial diagram demonstrating an electrode cuff for direct measurement of bilaterally symmetric segments of a patient anatomy;

FIG. 5 is a flow chart demonstrating an assessment routine for identifying injuries or abnormalities;

FIG. 6 is an illustrative diagram demonstrating levels of severity of injury that may be distinguished by the assessment routine described in FIG. 5;

FIG. 7 is a line chart demonstrating a differential impedance response of a patient over a recovery period;

FIG. 8A is a bar chart demonstrating a stratification of combined differential resistance measures or levels among injuries of varying levels of severity;

FIG. 8A is a bar chart demonstrating a stratification of combined differential reactance measures or levels among injuries of varying levels of severity;

FIG. 9 is a data table demonstrating a percent bilateral difference of bioimpedance data for subjects with anterior cruciate ligament injuries over a diagnosis, treatment, and recovery timeline;

FIG. 10A is a data table demonstrating a percent bilateral difference of bioimpedance data for subjects with a variety of ankle injuries;

FIG. 10B is a data table demonstrating a percent bilateral difference of bioimpedance data for subjects with a variety of hamstring injuries;

FIG. 11 is a flow chart demonstrating a method for completing an assessment of bioimpedance data with a detection model;

FIG. 12 is a flow chart demonstrating an assessment method that implements bioelectrical impedance in conjunction with a trained model for a preliminary condition determination; and

FIG. 13 is a block diagram showing a monitoring system providing a connected infrastructure for multiple bioimpedance measurement or monitoring devices.

### DETAILED DESCRIPTION OF THE PREFERRED EMBODIMENT

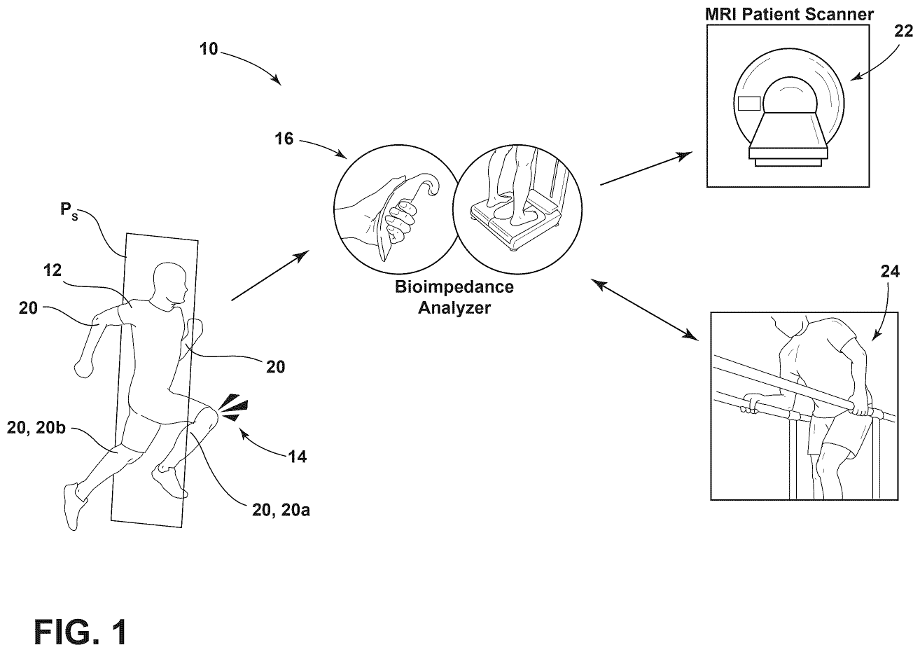

For purposes of description herein, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations, except where expressly specified to the contrary. It is also to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

The terms “including,” “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises a . . . ” does not, without more constraints, preclude the existence of additional identical elements in the process, method, article, or apparatus that comprises the element.

Referring generally to FIG. 1, a pictorial process diagram is shown demonstrating general procedural steps defining a method 10 for assessment and treatment of a patient 12 from an injury 14 or various conditions that may be associated with conditions that may result in pain and/or discomfort. In general, the assessment method 10 may utilize a bioimpedance analyzer 16 to determine the existence and relative level of severity of the injury 14 or abnormality experienced by the patient 12. In addition to conditions that may be reported by the patient 12 or suspected by a qualified professional based on examination or health history, the methods and devices described may provide for indications of tissue conditions that may lead to injuries or exacerbate existing conditions. For example, pain may be associated with various conditions, only some of which may be detrimental or present risk of further injury. However, soreness and tightness may be prevalent regardless of the underlying cause of the discomfort. Further, one or more underlying conditions may be present in the patient 12 but unnoticed. In such cases of latent or unreported conditions that may lead to injury, the methods described associated to the bioimpedance analyzer 16 may present indications of potential injuries, tissue conditions, or abnormalities that may indicate the potential for more significant injuries or deterioration in tissue quality that may be reversed or avoided by early intervention.

Though discussed primarily in reference to the injury 14 and exemplified as sports injuries, it shall be understood that various physical or medical conditions, abnormalities, and/or musculoskeletal disorders may similarly be detected and gauged in severity by utilizing steps similar to those discussed in reference to the assessment method 10. In some cases, the conditions described as injuries may result from a variety of health conditions that may include neurological disorders (e.g., muscular dystrophy). Further, though discussed in reference to a human patient, the methods and devices discussed herein may be implemented to treat various animals with similar relevant pathology including, but not limited to, horses, dogs, cats, livestock, and/or more exotic orders of mammals (e.g., zoo animals). Accordingly, the assessment methods and equipment discussed herein may be generally applicable to detect various defects associated with the patient 12. For clarity, the various conditions (e.g., induced or congenital), defects, abnormalities, and/or injuries are generally referred to as injuries hereinafter.

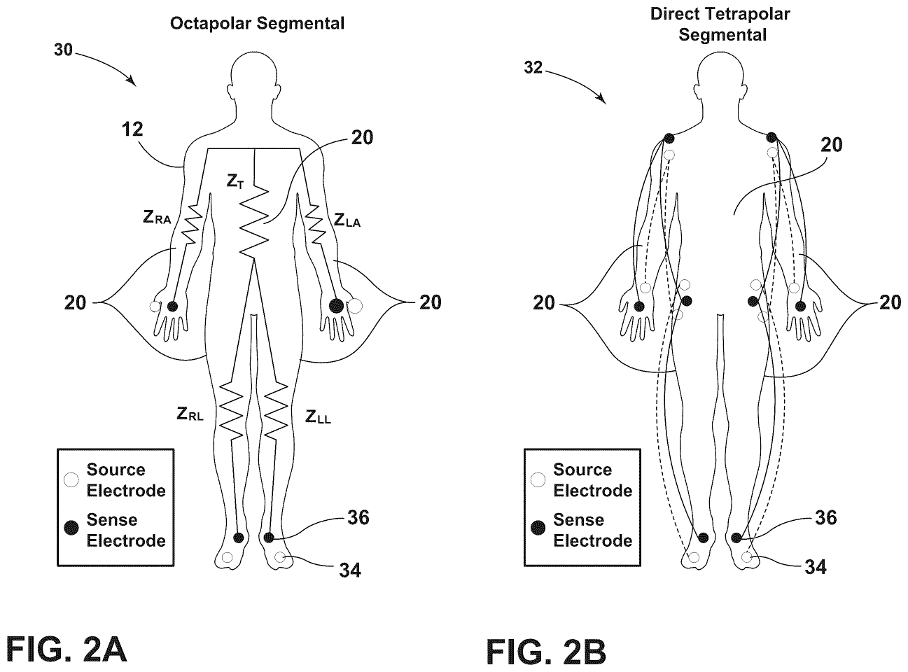

In various implementations, the method 10 may utilize the bioimpedance analyzer 16 to provide a segmental bioimpedance analysis to the patient 12 that may be applied over the whole body and/or across specific segments. Further discussion of the specific equipment and associated methods of bioimpedance analysis are discussed in reference to FIGS. 2-4. In general, the bioimpedance analyzer 16 may apply an excitation signal at a known frequency (e.g., 1-1000 kHz, 5-250 kHz, or commonly 50 kHz) and detect characteristic signals representative of an impedance Z extending along various segments 20 of the patient 12. In the example shown in FIG. 1, segments 20 may correspond to a right leg 20a and a left leg 20b forming bilateral anatomical segments 20 across a sagittal plane P_{S}. Additional segments may correspond to opposing arms, portion of the trunk, and various specific underlying segments (e.g., knees, elbows, hips, etc.). Based on a comparative analysis of the characteristic signals recorded by the bioimpedance analyzer 16, the injury 14 may be attributed to the segment 20 (e.g., the right leg 20a) demonstrating a reduced or changed bioelectrical impedance. As later discussed in various examples, the comparative analysis may be evaluated based on a bilateral comparison of comparable tissue symmetrically across the anatomy and/or a comparison of the response of tissue of the patient 12 measured over various time intervals. Such time intervals may correspond to milestones associated with an injury or strain, recovery/rehabilitation milestones and/or tests administered at various intervals or frequencies through training or recovery.

In addition to the identification assessment of the injury 14, a level of severity of the injury 14 may be identified providing an objective metric to guide a treatment plan for the patient 12. For example, following many injuries or indications of pain/dysfunction, practitioners may apply a variety of superficial examination procedures such as range of motion and tissue stress tests to attempt to identify a level of severity of an injury as reported by the patient 12. However, such examinations are highly subjective or often difficult to quantify. Further, many diagnostic techniques, such as advanced imaging equipment 22 (e.g., magnetic resonant imaging [MRI], computer tomography [CT] scans, etc.), are often delayed or inaccessible due to limited resources and cost limitations. By applying the method 10 to determine the relative impedance of the segments 20, the disclosure may provide for an objective indication of the presence and relative level of severity of the injury 14, such that physicians and healthcare professionals may assess or diagnose patient conditions and determine whether advanced treatment for the patient 12 is warranted.

In addition to conventional reactive treatment to reported discomfort or measurable deterioration in function, the method 10 and related assessment disclosed may be applied proactively to monitor tissue health before an injury is reported. As previously discussed, a subjective severity of pain or discomfort may vary depending on the patient 12 and may go unreported. Further, some pre-injury conditions may not even be associated with discomfort. By detecting early signs of inflammation or damage in the tissue of the patient 12 or subject that deviate bilaterally from a comparatively healthy corresponding tissue segment or differ from a historic measurement or series of measurements captured by the bioimpedance analyzer 16 over time as a comprehensive assessment plan, the devices and methods disclosed may provide early indications escalating deterioration in tissue, failures to maintain historic norms in recovery rate, and/or indications of latent or unreported conditions that may result in injury if not addressed. In this way, the methods, devices, and systems disclosed may provide early indications that may even prevent unnecessary injuries that could be avoided by simple precautions, such as rest.

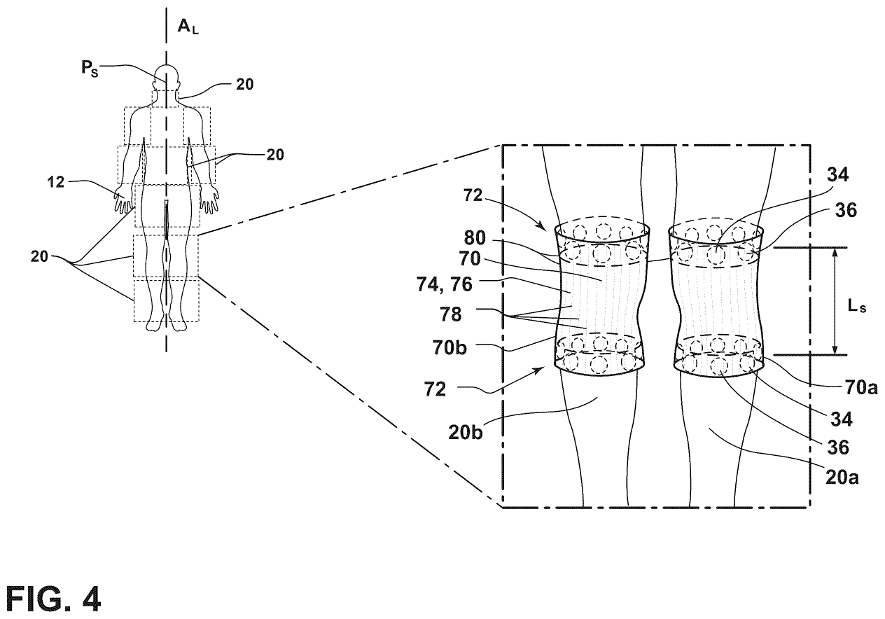

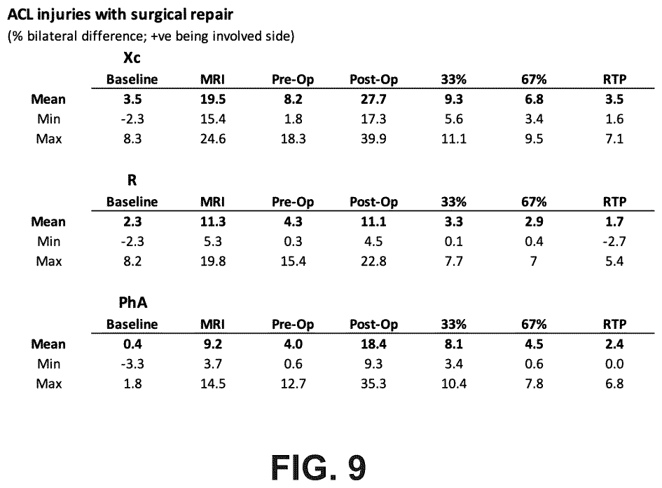

As demonstrated in the disclosed results, in some cases, the disclosure may provide for indicators that can be evaluated to determine conditions that may present an elevated chance of potential injury. Based on these indicators, instructions may be provided to adjust behavior, training techniques, intensity, timing, etc. of exercise or physical exertion to promote productive exercise and activity and dissuade or instruct against activities that may present elevated risk of injuries. For example, in various cases, the disclosed monitoring devices and methods may be configured to map a tissue condition profile in terms of the bioimpedance response of each patient in a patient profile. As discussed later in reference to a detailed training and recovery monitoring method in FIG. 9, the disclosure may provide for ongoing or periodic measurements of the bioimpedance of an individual monitored during a training or rehabilitation routine to determine corresponding measures of a baseline as well as characteristics or thresholds associated with peak strain, fatigue, and resulting soreness/recovery times. Such bioimpedance measurements may be captured via a segmental analysis as demonstrated in FIGS. 2A and 2B, measured via one or more targeted monitoring devices (e.g., electrode cuffs, electrode arrays, etc.) as demonstrated and described in reference to FIG. 4.

Based on the periodic or prescribed monitoring, the characteristic response of each individual may be monitored over time. As part of an ongoing monitoring routine, attributes of the bioimpedance response comparison may provide indicators to a qualified professional that the conditions measured in a routine may be indicative of a heightened risk of future deleterious tissue responses or more significant injuries. Such risk conditions may be detected based on variations in the bioimpedance response exceeding a threshold outside normal historic variations for the individual. Similarly, the data for each individual and/or a population of users/patients may be mapped to identify trends, thresholds, and corresponding conditions indicative of heightened risk conditions as part of a statistical model or trained model. In such cases, the comparison of a routine measurement of a user may be compared to historic monitoring data, for the individual or based on many indicators that may be common among a monitored population or subject database, to identify conditions indicative of overloading or potential injury. In response to such indications, a computerized monitoring system may provide one or more alerts or indications of the potential risk condition as well as corresponding suggestions to avert the potential risk.

Similarly, in a medical treatment context, the disclosed method 10 may provide for a powerful tool to provide a preliminary assessment of various tissue conditions. For example, the method 10 may be effective in providing guidance to determine whether the severity of an injury is sufficient to necessitate diagnostic imaging (e.g., MRI), advanced therapeutic or surgical intervention. For example, if the bioelectrical impedance indicates a level of severity exceeding a threshold, the clinician may be directed to submit the patient 12 for additional scans via the advanced imaging equipment 22. However, in cases where the level of severity is considered less so and initial assessment determines surgical intervention is not necessary (e.g., minor to severe sprains, etc.), the physician may confidently prescribe therapeutic intervention (rehabilitation, physical therapy) 24 to properly treat the patient 12. Accordingly, the method 10 and equipment discussed herein may provide for improved assessment and treatment of patients with various injuries while also limiting unnecessary expenses associated with advanced imaging equipment 22. Further, if rehabilitation 24, an intervention plan, or similar nonsurgical treatments are prescribed, the physician may monitor the impedance response associated with the injury 14 over time to assist in adjusting the intervention plan 24 to assist the patient 12 to return to normal activity over an expedited timeframe. These benefits, as well as other features and aspects of the assessment method 10, are further described in the detailed figures and description that follows.

Referring now to FIGS. 2A and 2B, electrical schematic diagrams of an octopolar segmental bioelectrical impedance model (OS-BIA) 30 and a direct tetrapolar segmental bioelectrical impedance analysis (TS-BIA) 32 are shown illustrating a plurality of source electrodes 34 and sense electrodes 36 distributed over the anatomy of the patient 12. As demonstrated in FIG. 2A, each of the bioelectrical impedance analysis methods OS-BIA 30 and TS-BIA 32 may be applied to detect a current resulting from an impedance Z extending along each of the segments 20 of the patient 12. In the example shown, the impedance segments measured by the analysis methods include the following: right arm impedance Z_{RA}, left arm impedance Z_{LA}, right leg impedance Z_{RL}, left leg impedance Zu, and a trunk impedance Z_{T}. In operation, the OS-BIA 30 utilizes fewer source electrodes 34 and sense electrodes 36 and measures the current flow and voltage across each of the source electrodes 34 and sense electrodes 36 to estimate the impedance Z of each of the segments 20. In contrast, the TS-BIA 32 incorporates a source electrode 34 and sense electrode 36 pair across each of the segments 20 and provides for direct measurement of the corresponding impedance Z. In each case, research has demonstrated that accurate estimates of the bioelectrical impedance of each of the segments 20 may be estimated.

In some implementations, the disclosure may provide for specialized cuffs or sleeves that may assist in conductively coupling the electrodes 34, 36 to the patient 12 or subject. For example, a wearable sleeve is shown in FIG. 4 demonstrating an array of electrodes 70 separated across opposing segments 20 of the patient. Alternatively, or for additional testing, a sleeve may be worn by the patient 12 that is wrapped about the hands or feet and positions the electrodes 34, 36 as shown in FIG. 2A. In this way, the patient 12 may be positioned on a table or testing surface with the limbs positioned with adequate separation (e.g., from each other and the trunk) for consistent positioning among subjects. The coupling of the electrodes 34, 36 to the patient 12 with the sleeves may limit variations in testing and provide for consistent measurements of the impedance Z with the patient 12 or subject positioned on the testing surface (e.g., laying in a prone or supine position).

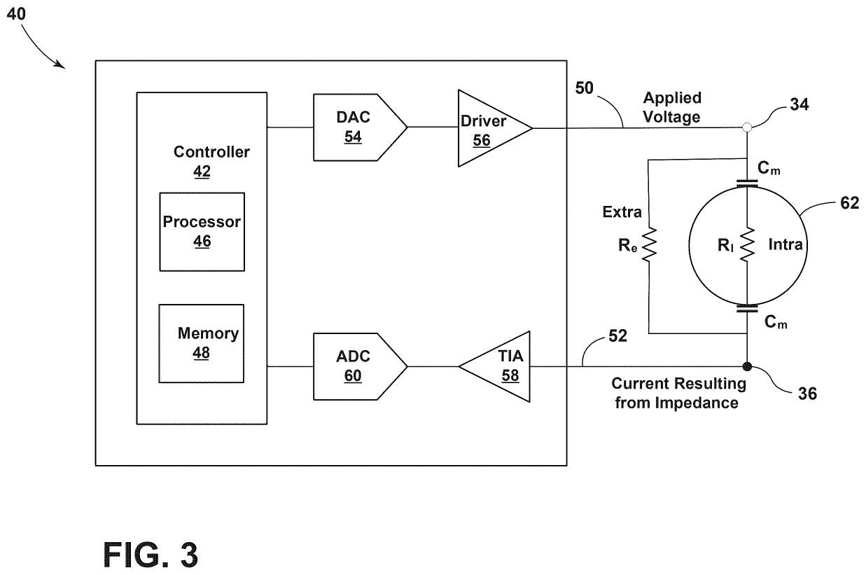

Referring now to FIG. 3, a simplified block diagram of an assessment system 40 is shown demonstrating the exemplary operation of a controller 42 and detection circuitry 44 that may be utilized to enable the operation of the bioelectrical impedance analysis (analyzers 30, 32) as discussed herein. In general, a controller 42 may comprise one or more processors 46 and a memory 48 that may store various routines and processing steps that may be implemented to output an excitation signal 50 from the source electrodes 34 and interpret a characteristic signal 52 detected at the sense electrode 36. More specifically, the controller 42 may communicate a control signal to a digital-to-analog converter 54, which outputs an analog control signal to a driver 56 to deliver the excitation signal 50 to each of the source electrodes 34. The current resulting from the bioelectrical impedance Z is detected by the sense electrodes 36 and supplied as a characteristic signal 52 to a transimpedance amplifier (TIA) 58, which is further supplied to a high-resolution analog-to-digital converter (ADC) 60. The resulting digital signal communicated by the ADC 60 is processed by the processor 46 to extract the information required for the bioelectrical impedance analysis. More specifically, the data from the ADC 60 is processed to detect the amplitude and timing of the characteristic signal 52 and compare the characteristic signal 52 to the excitation signal 50. Based on the shift in the waveform associated with the characteristic signal 52, the phase angle PhA associated with the resistance R and the reactance X_{C} may be determined and calculated for each of the segments 20 of the anatomy of the patient 12.

In various implementations, the excitation signal 50 may correspond to a frequency of 50 kHz, which has been determined empirically to provide meaningful feedback in relation to both an intracellular impedance and an extracellular impedance. As shown in FIG. 3, the intracellular impedance may be a combination of an intracellular resistance R, within a cell membrane 62 and a membrane capacitance CM attributed to the walls of the cell membrane 62. The total impedance Z may further incorporate the extracellular resistance RE associated with the fluid surrounding the cell membrane 62. The frequency of 50 kHz associated with the excitation signal 50 presents a balance of accurate detection of a fluid mass and body cell mass in combination for each of the segments 20, which provides information for both the intra and extra-cellular components of the tissue in each segment 20. Lower frequencies (e.g., approximately 1 kHz to 50 kHz) may primarily provide characteristic signals 52 representative of the extracellular resistance RE, while higher frequencies (e.g., approximately 50 kHz to 200 kHz) may pass more readily through the walls of the cell membrane 62 and primarily provide insight into the intracellular resistance RI and membrane capacitance CM. Accordingly, frequencies of approximately 50 kHz were applied in the generation of the test data later presented in this disclosure and provided more readily identifiable shifts representative of soft tissue damage.

While the frequency of the excitation signal 50 is described as corresponding to a 50 kHz control signal, the frequency may vary depending on the objectives of the system and may vary from approximately 1 kHz to 1 MHz. Accordingly, the assessment system 40 may be implemented in various applications. Circuits and products that capture bioimpedance information may include body composition analyzers that may be applied to determine body fat levels, lean mass, and hydration levels. One product is provided by Inbody as Model 770. Additionally, bioimpedance analyzer circuits may be purchased as integrated circuit packages, for example AD5940 from Analog Devices.

Referring now to FIG. 4, an illustrative diagram is shown demonstrating an electrode cuff 70. As shown, the electrode cuff 70 may correspond to a pair of electrode cuffs 70a, 70b that may be fitted about corresponding segments 20 of the patient 12. As previously discussed, the segments 20 may be bilaterally symmetric across the sagittal plane P_{S} such that similar anatomical features are present for comparison. In the example shown, the electrodes 36 may form opposing electrode arrays 72, including a plurality of source electrodes 34 and sense electrodes 36. As shown, the source electrodes 34 are interposed between the sense electrodes 36. In this configuration, the electrodes 34, 36 may be alternatively activated across a predetermined length or segment length L_{S} extending between the opposing electrode arrays 72 on each of the cuffs 70. The spacing between the opposing electrode arrays 72 or opposing electrodes may be fixed to the segment length L_{S} for each of the first electrode cuff 70a and the second electrode cuff 70b to ensure that the distance of each of the corresponding segments 20 of the patient 12 are of approximately equal distance. In this way, the electrode cuffs 70, when positioned over opposing symmetric segments 20 of the patient 12, may provide for accurate readings by limiting variations associated with inconsistent spacing of the opposing electrode arrays 72.

The opposing electrode arrays 72 may be interconnected by connecting material 74 that may correspond to a semiflexible or elastic connecting sheath 76. Accordingly, the connecting material 74 may correspond to a flexible material, such as Lycra or elastic, or other common textile materials, such as cotton or polyester, combined with synthetic rubber, to provide a comfortable, secure fit over various proportions of the anatomy. To ensure the segment length L_{S} is consistent across the symmetric segments 20 of the anatomy of the patient 12, the connecting material 74 may be longitudinally reinforced by one or more rigid strips of material 78 or otherwise woven or reinforced to allow stretching to conform to the segments 20 while limiting stretching along the longitudinal axis A_{L}. In this way, the segment length L_{S} may be maintained while further allowing the connecting sheath 76 to conform to the various segments 20 of the patient 12.

In operation, the electrode cuffs 70 and the corresponding electrode arrays 72 may apply multiple excitation signals 50 across opposing source electrodes 34 and sense electrodes 36 spaced apart by the segment length L_{S}. The resulting characteristic signals 52 for each of the individual electrode cuffs 70 may first be compared to other signals from the same electrode cuff (e.g., compare characteristic signals for a left or first cuff 70a) to calculate the corresponding impedance Z. In this way, the detection method may ensure that the measurement of the impedance Z is repeatable and accurate among the opposing electrode pairs of source electrodes 34 and sense electrodes 36 for each of the opposing segments 20a, 20b.

For example, the controller 42 previously introduced in FIG. 3 may communicate multiple excitation signals 50 and detect corresponding characteristic signals 52 for each of a left segment 20a and a right segment 20b. Based on the impedances Z for the left segment 20a and the right segment 20b, the controller 42 may compare the left segment impedance value Z as well as the right segment impedance value Z_{R} to signals captured over the same segment 20 to calculate the corresponding left and right impedances Z_{L}, Z_{R} accurately and omit or otherwise account for outliers detected among the characteristic signals 52. Once the left and right impedance values Z_{L}, Z_{R} for each of the left segment 20a and the right segment 20b are confirmed, the controller 42 may continue the assessment method 10 by comparing the left and right segment impedances Z_{L}, Z_{R} to calculate a difference or percent difference and assess the severity of the injury 14 according to the difference in the impedances Z_{L}, Z_{R}. In this way, the assessment method 10 of the disclosure may provide for improved assessment and proposed treatment options that may serve as an adjunct to inform a clinical diagnosis to improve patient care.

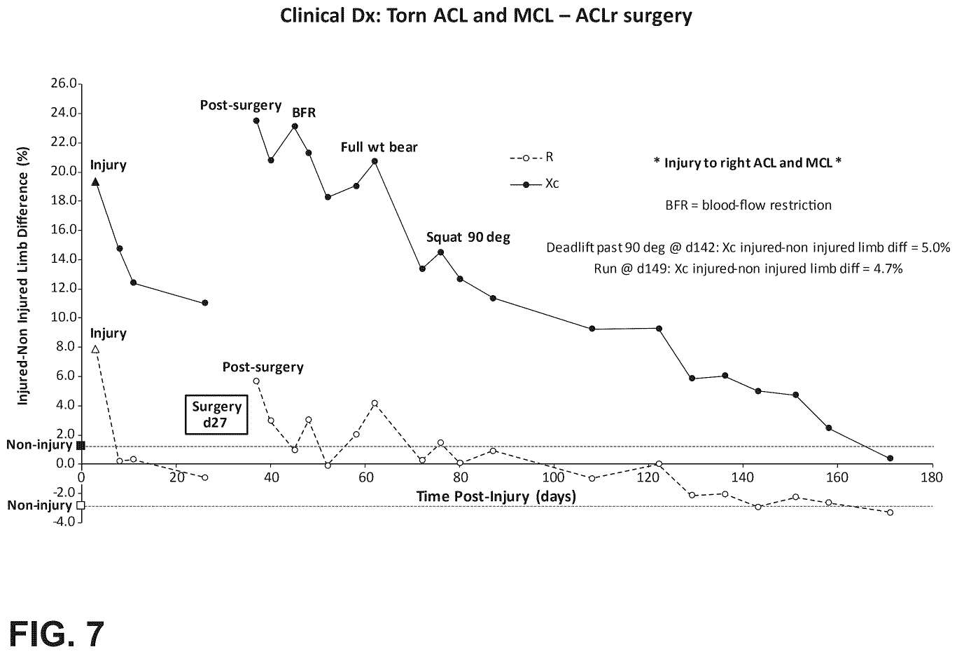

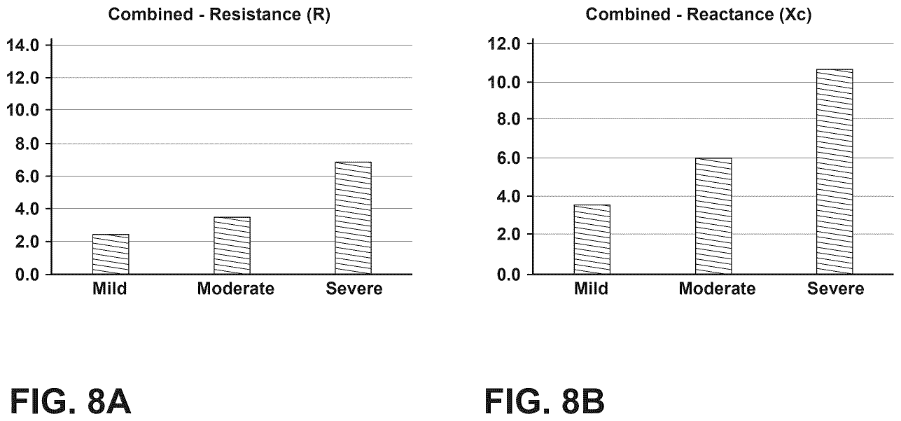

Though the impedance Z is primarily described in reference to the method 10 for assessment, the underlying resistance R and/or capacitive reactance X_{C} associated with the impedance Z may individually be evaluated. Additionally, the phase angle PhA may be evaluated. The phase angle PhA may correspond to a measure of body cell mass and hydration, that may be serve as another indicator of potential soft tissue or tendon injury calculated as the arctangent of reactance (X_{C}) divided by resistance (R), multiplied by (180°/π). In some implementations, the reactance X_{C} associated with the impedance Z may report physiological differences associated with the injury 14 with a higher magnitude than the resistance R. However, as further demonstrated in the test data shown in FIGS. 7 and 8, both the resistance R and the reactance X_{C} associated with the impedance Z may accurately correlate to the presence and relative severity of the injury 14 as well as a tissue response to the intervention of treatment of the patient 12.

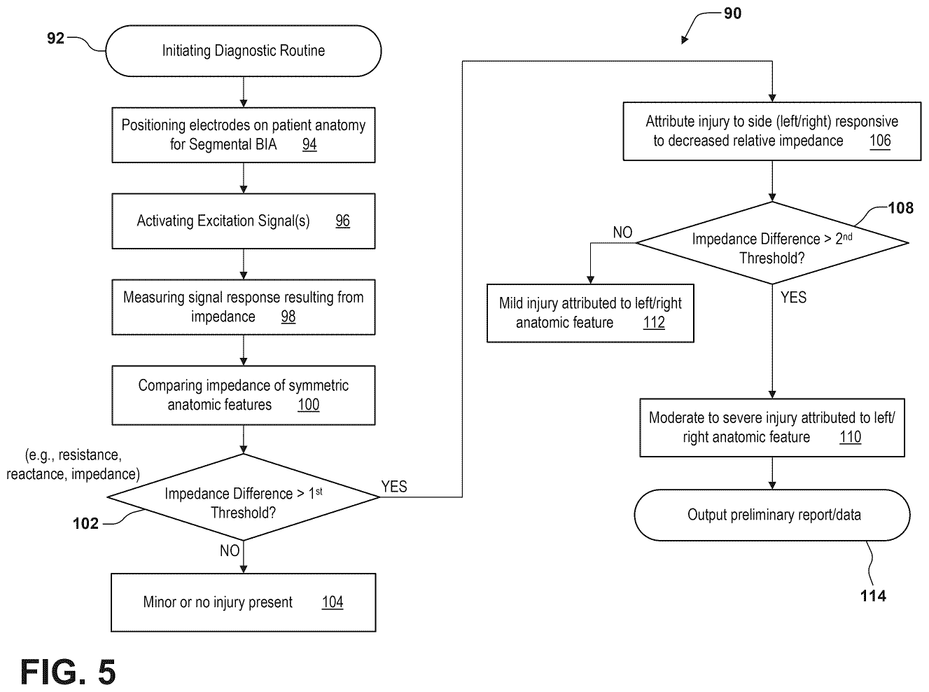

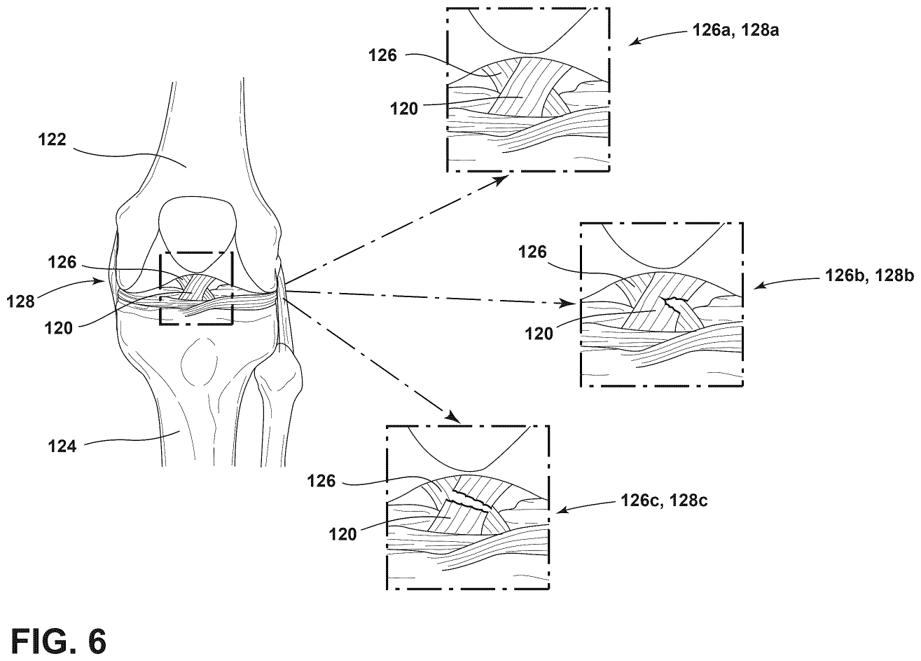

Referring now to FIGS. 5 and 6, a flow chart is shown demonstrating an exemplary assessment method 90 (FIG. 5) and corresponding examples of injuries (FIG. 6) associated with the method 90. As previously discussed, the assessment method 90 may begin by first initiating the assessment routine 92 by positioning the electrodes 34, 36 on the anatomy of the patient 12 (94). As previously discussed, the OS-BIA 30, TS-BIA 32, and/or the individual cuff placement demonstrated in FIG. 4 may be applied to position the electrodes in step 94. Following the positioning of the electrodes, the controller 42 may activate the excitation signal 50 in step 96 and measure the signal response of the resulting characteristic signals 52 in step 98 to calculate the impedance Z. Once calculated, the impedances Z of each of the bilaterally symmetric segments 20 may be compared to determine an impedance difference of percent impedance difference (100). In step 102, the method 90 may compare the percent impedance difference for each of the segments 20 to a first threshold. For example, if a percent difference between the impedance values Z_{L}, Z_{R} is less than 4% for the reactance X_{C}, the method 90 may determine that there is a low likelihood of injury or that no injury is present that requires advanced surgical intervention in step 104. If the impedance difference is greater than the first threshold (e.g., 3% to 6% difference in X_{C}), the method 90 may continue to step 106 and output a communication that indicates that there likely is a mild to severe injury to the corresponding side of the patient 12 demonstrating the decreased relative impedance.

Once an injury is attributed to one of the laterally opposing segments 20, the controller 42 may continue to compare the impedance difference to a second threshold in step 108. For example, if the percent impedance difference between the left and right segments 20a, 20b indicates a percent difference in the reactance X_{C} that exceeds a second threshold (e.g., 5% to 12% difference in X_{C}), the controller 42 may determine the likely presence of an injury or compromised tissue condition in step 110 as a moderate to severe injury. Alternatively, if the impedance difference, in this case, the percent difference in the capacitive reactance X_{C}, is less than the second threshold in step 108, the controller 42 may output an indication that a mild injury is attributed to the corresponding segment 20 of the patient 12 in step 112. Based on the relative severity of the injury, the qualified professional can readily determine subsequent steps for treatment.

Though discussed in reference to specific magnitudes of impedance differences, it shall be understood that the specific values associated with the measured impedances Z (e.g. resistance R and reactance X_{C}) may vary in magnitude or relative scale depending on the specific procedures, equipment, electrode positions, or other factors. However, it shall be understood that comparisons of the resulting impedance measurements will still vary among conditions or injuries of differing levels of severity. Accordingly, the methods, devices, and assessment systems discussed herein are not limited to operation described in the specific operating ranges described. Instead, the examples provided are instructive of the methods of comparative analysis associated with the bioimpedance Z as generally applied to support the disclosed methods for assessment.

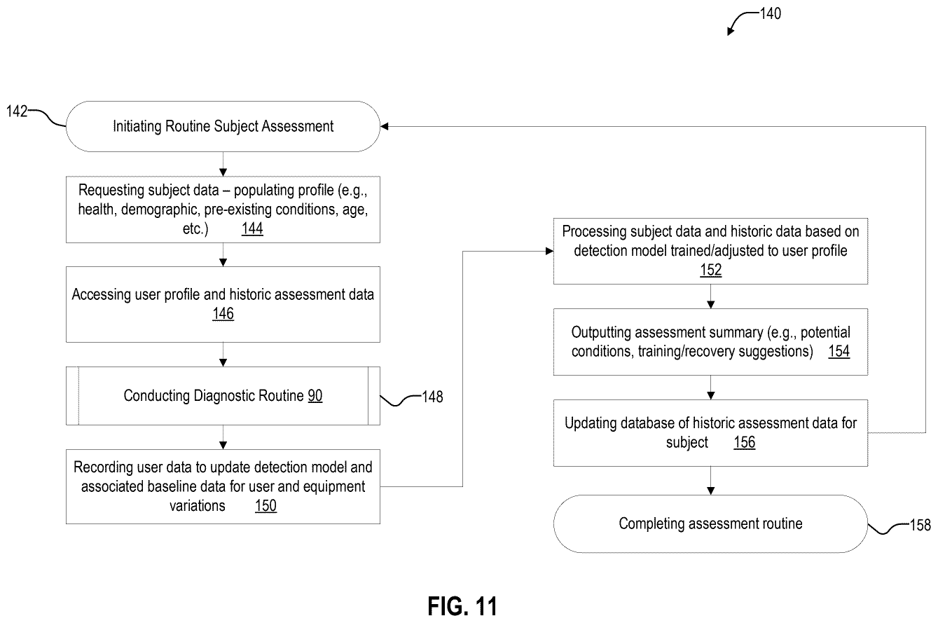

As discussed herein, the thresholds may correspond to predetermined percentages or differences in the impedance values Z_{L}, Z_{R} that may serve as indicators of the relative severity of the inflammation or symptoms associated with an injury or tissue condition. As later discussed in reference to comparative measurements of the impedance values over time or in comparison to a baseline for the patient 12 or subject, the thresholds may similarly correspond to differences in impedance compared to values associated with healthy tissue for the patient 12 that may be previously measured. Further, the thresholds or corresponding values/levels/measurements associated with the impedance data for the patient 12 or subject, may vary depending on various factors, including subject data, injury or condition data, as well as additional sensor data as discussed in further detail in steps 144, 146, 152, and/or 154 of the method 140 as later discussed in reference to FIG. 11. Accordingly, the thresholds discussed herein may correspond to indicators attributed to the location (e.g. segment 20, limb, joint, etc.) of the injury 14 or potential injury, the condition of the patient, the nature of the injury or events leading to the injury, and additional sensor data that may provide assessment data to inform the preliminary assessment of a condition of the tissue of the patient 12 or subject.

Following the operation of the method 90, the controller 42 may output a preliminary assessment report and corresponding impedance data in step 114 that may provide a preliminary assessment of both a type and severity of the injury 14 as well as supplemental information that may assist in determining the clinical steps that should be administered. Though specific ranges of the percent difference of the reactance X_{C} are described in reference to the first threshold in step 102 and the second threshold in step 108, it shall be understood that the magnitude of the differences calculated for the corresponding characteristic signals 52 may largely be attributed to the specific hardware or equipment utilized to perform the method 90. For example, the operation of the various components of the detection circuits (e.g., the detection circuitry 44) may vary in magnitude, amplifier gain, and various other attributes that may elevate the magnitude and/or adjust the accuracy of the interpretation of the characteristic signals 52. Accordingly, the percent differences described in reference to the first threshold, the second threshold, and various other metrics discussed herein are for the purposes of example to support the operation of the methods and equipment described herein.

Referring now to FIG. 6, an exemplary injury is described in reference to an anterior cruciate ligament (ACL) 120 shown relative to a femur 122 and a tibia 124 of the patient 12. As discussed in reference to the method 90 of FIG. 5, in step 104, if no injury is present, the ACL 120 may be deemed or preliminarily assessed to be minorly sprained but otherwise in an in-tact or healthy condition 126a as exemplified by the healthy ACL 128a. If the impedance difference in step 102 exceeds the first threshold, the injury accessed in step 106 may be attributed to a moderately injured condition 126b (e.g., partially torn or otherwise damaged). For clarity, the assessment resulting from percent difference in the impedance Z may not necessarily identify the specific nature of the condition or injury. Testing has shown that the differential impedance analysis disclosed accurately indicates the segment 20 and relative severity of the injury 14. Additionally, the severity of the injury as indicated by the variations in impedance may allow the type of injury or condition to be classified to a list of likely injuries or injury types that may be associated with a difference in impedance Z detected bilaterally across the sagittal plane P_{S} or in comparison to a baseline associated with the patient in a prior state of comparatively good health. Such a classification may be determined based on an equation or lookup table that may attribute classes or injury or conditions to the corresponding levels of variation in impedance. With this information, a qualified provider may readily ascertain a region that may be associated with a compromised tissue condition that may be associated with pain or otherwise go unnoticed by the patient 12. Based on the preliminary information associated with the impedance Z measurements as discussed herein, a qualified provider may detect a possible tissue condition or injury and further surmise or estimate a severity of the injury 14 or tissue condition. This information may inform or lead to additional testing or examination that may support a clinical diagnosis by a qualified medical professional.

For example, in the example shown, the moderate injury condition 126b, when considered in combination with the location, pain, and range of motion, may inform a provider that the injury corresponds to a partially torn tendon 128b as attributed to the ACL 120 or determine that another soft tissue injury is apparent (e.g., a partial tear of the medial collateral ligament [MCL] 128). Categorizing the level of severity in such determinations is imperative to the treatment of the patient 12 because the subjective symptoms associated with severe injuries may be very similar to sprains that may not require additional surgical intervention. Many preliminary examinations may fail to identify severe injuries or overtreat minor injuries. Based on the impedance difference data associated with the method 90, a healthcare provider may improve the confidence of the assessment to improve patient care as well as prescribe the appropriate course of treatment. Accordingly, the methods and systems disclosed may be highly effective in improving patient care while limiting unnecessary expenses often associated with advanced scanning that may not be necessary for less severe injuries.

Still referring to FIGS. 5 and 6, the method 90 may further distinguish a level of severity among confirmed injuries. For example, if the method 90 indicates that the percent impedance difference exceeds the second threshold in step 108, the method may report a severe injury 126c in step 110. For example, the injury 14 may correspond to a completely torn tendon, muscle, or ligament condition 128c or other similar soft tissue injuries (e.g., muscle tear, ligament tear, etc.). As previously described, when combined with range of motion examinations as well as other forms of inspection or examination of the patient 12, the levels of severity identified by the method 90 may provide considerable insight into the status and necessary treatment that should be provided to the patient 12 to improve care and therapeutic intervention.

Referring now to Table 1, sample test results captured via the OS-BIA 30 are shown relative to corresponding clinical assessment by healthcare practitioners based on preliminary examination techniques. In particular, the data demonstrated in Table 1 confirms that each case of severe injury, as confirmed via MRI diagnosis, corresponds to an elevated percent difference in the capacitive reactance X_{C} in excess of at least 10% to 12% and generally in excess of 15%. Accordingly, each documented case investigated that included a bilateral percent difference in the capacitive reactance X_{C} in excess of the second threshold in the clinical study corresponded to a severe injury that required MRI scanning. Additionally, mild injuries, such as injury number 8, corresponded to the percent difference in capacitive reactance X_{C} in excess of the first threshold or greater than 3% to 5% but less than the elevated 10% to 15% difference in the bilateral capacitive reactance X_{C}. Accordingly, the assessment method 90 attributing the severity of injuries to different thresholds or ranges of the percent difference in impedance Z of the segments 20 was confirmed to accurately identify injuries and distinguish injuries among multiple levels of severity.

TABLE 1
Accumulated wb-BIA data for ACL injuries

| | | Clinical | | wb-BIA | |
|---|---|---|---|---|---|
| Injury | Initial Clinical | Diagnosis | Initial | Bilateral | wb-BIA |
| No. | Impression | (from MRI) | vs. MRI | Diff. X_{c} | vs. MRI |
| 1 | Severe ACL tear | Severe ACL tear | ✓ | 15.4% | ✓ |
| 2 | Severe ACL tear | Nothing present | x | 0.6% | ✓ |
| 3 | Severe ACL tear | Bone contusion | x | 0.9% | ✓ |
| 4 | Severe ACL tear | Severe ACL tear | ✓ | 17.3% | ✓ |
| 5 | Severe ACL tear | Severe ACL tear | ✓ | 21.8% | ✓ |
| 6 | Severe ACL tear | Severe ACL tear | ✓ | 17.5% | ✓ |
| 7 | Severe ACL tear | Severe ACL tear | ✓ | 22.7% | ✓ |
| 8 | Severe ACL tear | Mild LCL & | x | 5.2% | ✓ |
| | | meniscus tear | | | |
| 9 | Severe ACL tear | Severe ACL tear | ✓ | 20.0% | ✓ |
| 10 | Mild MCL tear | Severe ACL tear | x | 16.9% | ✓ |
| 11 | Severe ACL tear | Severe ACL tear | ✓ | 24.6% | ✓ |

Finally, from Table 1, the differences between the initial clinical impression and the resulting diagnosis from the MRI demonstrate the improved accuracy of the bilateral impedance difference assessment methods 10, 90 relative to conventional clinical approaches. For example, in each of injuries 2, 3, and 8, severe injuries were initially diagnosed but were contradicted by the percent bilateral impedance difference reported. In each case, the clinical diagnosis from the MRI verified that the percent bilateral impedance demonstrated improved assessment capability over the observations of the healthcare provider. Further, in particularly problematic situations, such as injury 10 where a mild injury was first diagnosed, the bilateral impedance comparison indicated that a severe injury was actually present. Later, the clinical diagnosis from the MRI confirmed the severe injury initially reported by the bilateral impedance of 16.9% in excess of the second threshold from the method 90. In such cases, the patient 12 may be misdiagnosed with a mild injury 126a while a severe injury 126c is present that may be further exacerbated if not treated accordingly. Therefore, the accumulated clinical results associated with the accuracy of the bilateral percent difference comparison of the impedance Z as presented in the disclosed methods may provide for improved patient care by communicating objective data that may be used in combination with various preliminary examination methods to ensure that appropriate treatment and referral are provided to the patient 12.

Referring now to FIGS. 7, the capability of the methods 10, 90 are further verified by clinical data presented in a line chart demonstrating repeated bilateral impedance percent difference measurements captured during a recovery period for the patient 12. As shown, the resistance R and the capacitive reactance X_{C} are demonstrated as measured over periodic intervals of time throughout the recovery of the patient 12 and attributed to specific milestones. The percent difference in resistance and reactance as assessed by the methods 10, 90 as discussed herein are presented over a 180-day period following the initial injury. Both the reactance X_{C} and the resistance R demonstrate a spike in a percent difference associated with the injury that decreases until additional trauma is introduced as associated with the surgical procedure. Following the surgery after day 40, various rehabilitation events (e.g., weight bearing, squats to 90%) result in corresponding elevated impedances associated with the injured segment 20. Additionally, as a recovery metric, the patient 12 was able to return to physical exercise and run on day 149 when the percentage difference between the injured and non-injured limbs and corresponding segments 20a, 20b decreased below 5%. These results further demonstrate that the method 90 may be utilized to gauge the state of recovery of the patient 12 and provide suggestions for elevated levels of exercise and activity levels that may be used to support accelerated recovery schedules for therapeutic intervention or rehabilitation.

Referring now to FIGS. 8A and 8B, bar charts are shown demonstrating the stratification of the measured impedances Z in the form of the resistance measurements R and reactance measurement X_{C}, respectively. In each chart, the results shown demonstrate the combined average values for a percent difference in resistance R and reactance X_{C} identified by comparing a first segment (e.g., right leg 20a) to a bilaterally symmetric, second segment (e.g., left leg 20b) of each subject. The results shown in FIGS. 8A and 8B were compiled for several instances of a variety of injury types, including strained muscles, moderate to severe sprains, muscle or ligament tears of varying severity, etc. As demonstrated in FIG. 8A, the percent difference in resistance R associated with mild injuries is approximately less than a 2.3%. Further, the percent difference in resistance R associated with moderate injuries is between approximately 2.3% and 3.4%. Finally, the percent difference in resistance R associated with severe injuries is between 3.4% and approximately 6.8% or in excess of approximately 3.5%. Accordingly, based on these results a clinician may determine whether further examination, diagnostic testing, or treatment should be considered for the patient 12.

As shown in FIG. 8B, the combined average values for a percent difference in the reactance X_{C} is shown for injuries corresponding to those demonstrated in FIG. 8A. Accordingly, a primary observation is that the percent difference in the resistance R and the reactance X_{C} correlate and share the same trends in magnitude responsive to increasing levels of severity. Further, the magnitude of the percent difference observed for the reactance X_{C} versus the resistance R is greater, which suggests the changes in resistance R resulting from injury may be more subtle than changes in reactance. As shown in FIG. 8B more specifically, the percent difference in reactance X_{C} associated with mild injuries is approximately less than a 3.7%. Further, the percent difference in reactance X_{C} associated with moderate injuries is between approximately 3.7% and 6.0%. Finally, the percent difference in reactance X_{C} associated with severe injuries is between 6.0% and approximately 10.6% or in excess of approximately 6.0%. Similar to the percent difference in the resistance R, the percent difference in reactance X_{C} may provide meaningful insight into a level of severity of a tissue condition or injury that may assist in the care of the patient 12 or subject.

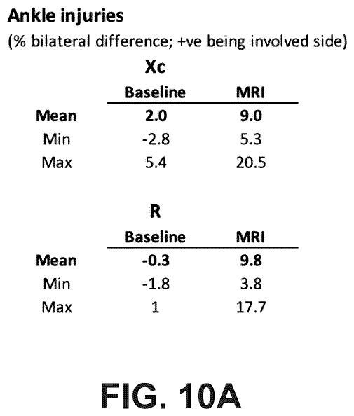

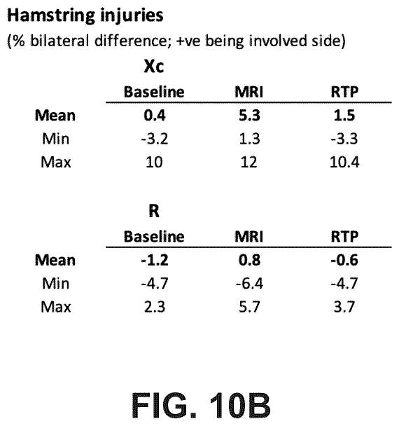

As shown in FIGS. 9, 10A, and 10B, additional sample data results from bilateral testing of the percent difference in resistance R and reactance X_{C} responsive to specific subject conditions are shown. The results are compared to clinical diagnostic procedures (e.g., MRI, etc.) that validate the correspondence of the bioimpedance to a spectrum of conditions that correspond to each physical milestone from injury to surgery intervention and recovery. Referring first to FIG. 9, impedance data for the reactance X_{C}, resistance R, and phase angle PhA is shown demonstrating the percent bilateral distance measured for a sample of ACL injuries over a period of time, including preinjury and post-surgical repair results. As demonstrated, each of the percent bilateral differences corresponding to baseline or healthy/normal conditions for subjects demonstrate a percent bilateral difference of approximately 4% or less. More specifically, the reactance X_{C} had a mean percent difference of 3.5%; the resistance had a mean percent difference of 2.3%; and the phase angle PhA had a mean percent difference of 0.4%. Following events corresponding to reported injuries, the percent bilateral difference was measured again at the time of a corresponding MRI. Following the injury and the corresponding imaging captured by the MRI, the mean reactance X_{C} increased to 19.5%; the resistance R increased to 11.3%; and the phase angle PhA increased to 9.2%. In each case, the corresponding percent bilateral difference associated with the post-injury diagnostic imaging for the subjects was multiplied by at least four times the magnitude of the baseline. Based on these results, test results having a variation of two times the base line in percent bilateral difference for any of the indicators (e.g., X_{C}, R, PhA) may suggest that a potential injury or conditions susceptible to injury. Accordingly, the percent bilateral difference identified for patients on average was an accurate indicator of the existence of the corresponding injury identified in the MRI imaging.

Following the diagnostic imaging, each of the subjects demonstrated decreases in the percent bilateral difference for each of the metrics while awaiting surgical operations. The corresponding decrease in bilateral difference may correspond to decreases in swelling associated with the tendon damage that may result from resting or immobilizing the corresponding tissue. Following the surgical operations, the percent bilateral difference increases again to a peak value for each of the metrics except for the resistance, which was approximately the same as when measured following the imaging with the MRI. The post-surgical or post-operative peak of the percent bilateral difference may be associated with the inflammation and tissue damage that may occur surrounding the original injury due to surgical access required for treatment at the injury site. Accordingly, each of the indicators for the ACL injuries on average provided meaningful indications of the corresponding health of the ligament of the subject.

Still referring to FIG. 9, the bilateral difference for each of the metrics was further monitored during the rehabilitation timeline at 33%, 67%, and 100% based on the return to play (RTP) date. The results associated with the recovery further demonstrate that the reactance X_{C}, the resistance R, and the phase angle PhA on average provide valid indications of the recovery of the associated tissue returning back toward the baseline in correlation with the recovery as determined by the associated athletic trainer. Of particular note, the mean or average values of the reactance X_{C} and the phase angle PhA provide meaningful indications of the corresponding tissue responses as verified by the subjects' athletic performance at each of their recovery milestones. Accordingly, the results of the bioimpedance analysis not only provide meaningful indications of injury but also may serve as accurate indicators of stages of recovery that may be evaluated to support various recovery and/or training programs.

As demonstrated in FIG. 9, the reactance X_{C}, the resistance R, and phase angle PhA each were reported as minimum values and maximum values as well as the mean values for the subjects. These values and the corresponding range of reported percent bilateral differences for each of the metrics demonstrate that even the outlying results, in most cases, provide accurate indications of the corresponding tissue condition. However, it is acknowledged that some of the results, for example, the negative results, may correspond to outlying data that may be improved over time based on improvements on the corresponding testing apparatuses. Accordingly, the status of the measurement equipment and improvements, some of which are described herein, have already and should continue to improve the accuracy of the corresponding results to provide indications of the corresponding tendon, ligament, and/or tissue condition.

Referring now to FIG. 10A, test results are shown for ankle injuries, reported as the bilateral percent difference in the reactance X_{C} and the resistance R. As shown by the limited data points available for ankle injuries, the baseline value for both the resistance X_{C} and the reactance R have a magnitude considerably lower than the results measured when a potential injury is reported. Further, the results demonstrate that the reported injury and the impedance data demonstrate a direct correspondence to clinically diagnosed injuries verified from imaging with the MRI. Accordingly, the results demonstrate that the reactance X_{C} and the resistance R measurements provide effective indicators of likely trauma and the increasing magnitude is shown to correlate to increased severity of the trauma. Of particular relevance, the data associated with the results in FIG. 10A corresponds to a wide variety of ankle injuries of varying severity, including sprains, contusions, and various associated trauma. Accordingly, the percent bilateral difference as reported with the resistance R and the reactance X_{C} may accurately indicate the existence of and relative severity of a wide variety of soft tissue injuries to tendons, ligaments, and other tissue that may be demonstrative of the associated joint health.

Referring now to FIG. 10B, resistance R and reactance X_{C} data is shown demonstrating the percent bilateral difference associated with a wide variety of hamstring injuries. Similar to the results previously discussed, the results reported for the reactance X_{C} provide particularly accurate indications of injury when compared to the baseline. However, again, favoring full disclosure and acknowledging potential improvements, the resistance for the limited sampling was not particularly informative between the baseline and the time of medical diagnostic imaging with the MRI. The data captured for the hamstring injuries may not currently reflect the expected accuracy of future testing apparatuses, primarily due to limitations of the test equipment available to capture the associated data. In particular, limitations in the positioning of the appendages of the subjects, particularly the leg placement, may have limited the clearance between the legs. Accordingly, it is believed that providing additional separation and/or conductive insulation between the legs may limit the outlying data and improve the results in the resistance R and reactance X_{C} measurements.

In yet another example, the condition of subjects following specific exercises were measured and evaluated to determine the percent difference in bioimpedance. In this case, the bioimpedance data represents the tissue response of bilateral arm segments and leg segments of 25 college baseball pitchers throughout a training regime. During the time monitored, the subjects did not experience any notable injuries. Accordingly, the data is not representative of any acute tissue trauma. The percent difference for the arm segments was reactance X_{C} of +2.3% and resistance R of +1.1%. As expected from the methodology discussed herein, the tissue on the side corresponding to the throwing arm (the positive side of the comparison) experienced elevated stress or tissue response relative to the non-throwing arm (the negative side of the comparison). The legs of the subjects showed similar signs of exertion with a reactance X_{C} of +1.9% and a resistance R of +1.0%. Based on the methodology disclosed, elevations in these results having a magnitude of 1.5-2.5 times the baseline could correspond to pre-injury indications that may warrant training, treatment, or diagnostic evaluations to prevent eventual injuries. Additionally, results having higher variations (e.g., 2-4 times the baseline) in magnitude may correspond to latent injuries or unreported injuries of the corresponding subjects.

Referring now to FIG. 11, a flowchart is shown demonstrating a method 140 for monitoring subjects for indications of overloading or injury. In general, the method 140 may provide for indications of risk conditions that may suggest changes in behavior and/or an exercise regimen. Beginning in step 142, the method 140 may begin by initiating a routine subject assessment. As discussed in various examples, the subject assessment may correspond to a bioimpedance analysis by a segmental approach and/or a direct measurement approach. Before capturing the diagnostic measurements, the method 140 may request subject data to populate a user profile (144). For example, the subject data may include health data, demographic data, preexisting conditions, injury location(s), age, training objectives, etc. Additionally, the subject data may include a profile indication that may be utilized to access a corresponding user profile and/or historic assessment data (146). Such data may be implemented to identify a baseline for the user and track variations in the percent bilateral difference of the bioimpedance detected over time.

In various implementations, the historic assessment data may provide for related data points from earlier impedance measurement that may be accessed for a direct comparison of the impedance for specific segments or measurements of the subject that may change over time. In such cases, the impedance analysis may include direct measurement comparisons as an alternative or in addition to the percent bilateral comparison described in various foregoing examples. In each case, following accessing the user profile, the method may continue to step 148 to conduct the diagnostic routine 90 as shown in FIG. 11. Following the diagnostic routine 90, the impedance data for the user may be recorded and used to update a detection module and/or associated baseline data in the user profile (150). In some examples, the equipment utilized to measure the subject may also be recorded in step 150. In this way, the data measured for one or more of the subjects may be anonymously compared to detect variations in amplitude and corresponding measurement results that may differ among different monitoring tools as well as resulting from repairs, maintenance, and/or modifications to the monitoring equipment. In this way, the results recorded for each of the subjects may be utilized in aggregate form to improve the accuracy of the monitoring results and detect potential issues in the monitoring equipment.

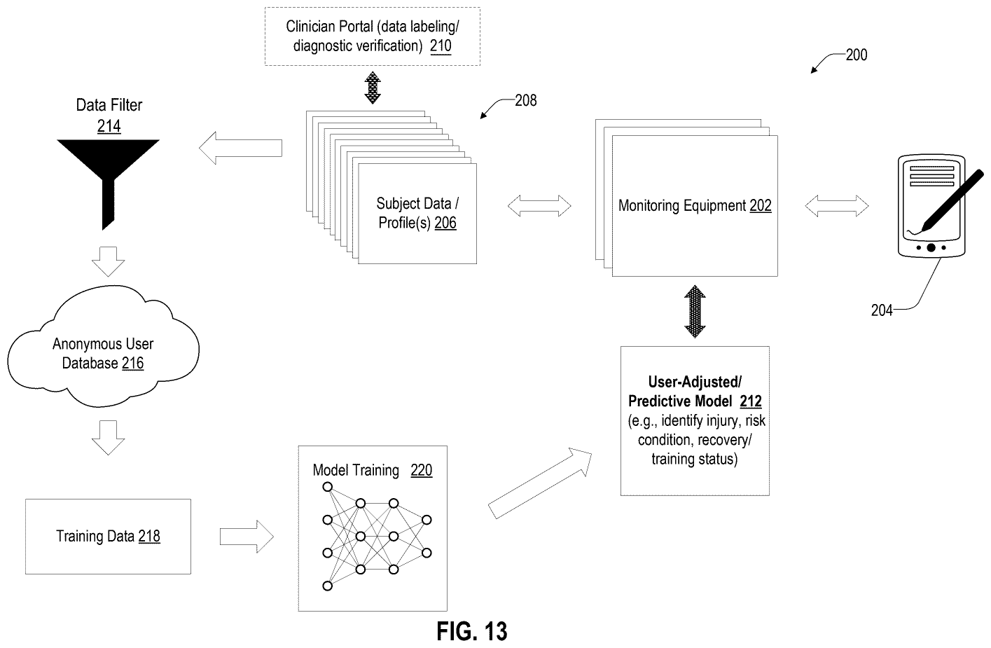

Still referring to FIG. 11, with the user data recorded in step 150, the method 140 may continue to step 152 to process the patient data and historic data based on the detection module and/or the baseline associated with the user profile. In various implementations, the detection module may correspond to an algorithm or trained model that may be configured to identify variations in the bioimpedance measured for various subjects and identify conditions that may be attributed to potential injuries or overloading conditions that may suggest an elevated risk of potential injuries. An example of a training model is further introduced and described in reference to the block diagram in FIG. 13. Once the subject data is processed, an assessment summary may be output in step 154 (FIG. 11), that may indicate potential injury conditions, as well as potential training and/or recovery suggestions. Once the subject data is reported, a database associated with the user profile may be updated for the subject (156), and the method 140 may be completed (158).

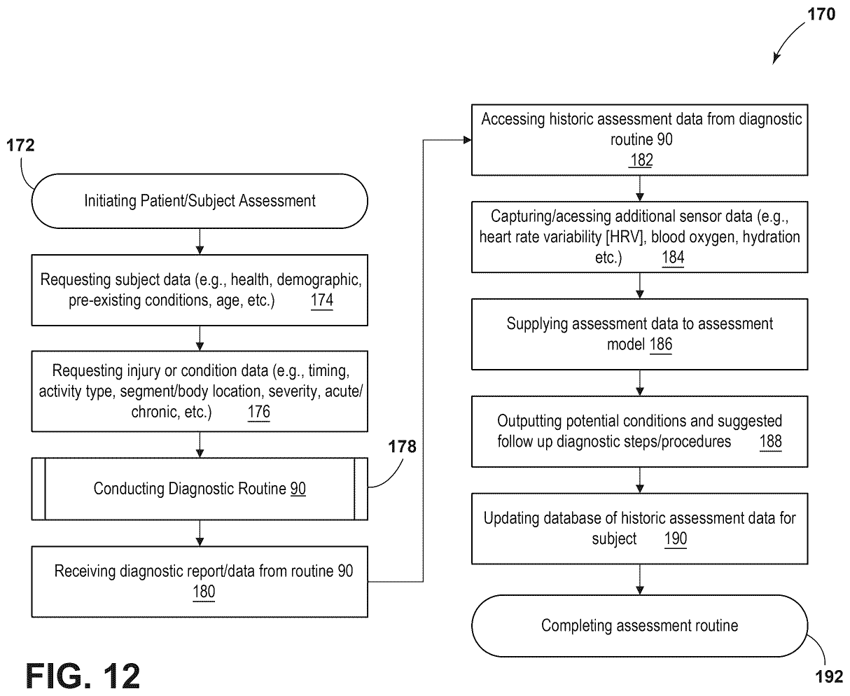

As shown in FIG. 12, following the initiation of the method 170 in step 172, the method 170 may begin by requesting patient data and injury or condition data in steps 174 and 176. For example, in step 174, the method may request a user (e.g., the patient 12 or healthcare provider) to enter subject data indicating the health statistics, demographic information, age, pre-existing conditions, etc. associated with the patient 12. Further in step 176, the method 170 may request injury or condition data that may include the timing, the activity type that resulted in the injury 14, the segment/body location, the perceived severity, and the acute or chronic nature of the injury 14. The information in steps 174 and 176 may be supplied to the patient via a series of prompts and may request information and include prompts/questions similar to those that follow.
Age and gender
Height and weight
Location: joint, anatomical area (muscle group etc.), directional terms (anterior, posterior, medial lateral, superior, inferior, etc.)
Grade level of discomfort (e.g., Likert scale)
Onset of injury (date/time)
Mechanism (acute, chronic)
What activity were you participating in (e.g., sport, exercise type, etc.)?
Duration/Chronicity or Pain/Condition: has the pain gotten better or worse?
Previous injury history?
Are you having difficulty moving the segment (e.g., limb) like normal?
Are you able to complete normal daily tasks with the current level of discomfort?
Do you currently have swelling in the affected area?
Do you currently have pain/discomfort in the affected area?

In addition to the subject data and the condition or injury data, the method 170 may access information including the bioimpedance data (e.g., an impedance difference for a segment 20) in steps 178 and 180. As previously discussed, the impedance data may provide an objective indication of the patient's physiological response to the injury 14, which may serve to improve the accuracy of the preliminary assessment provided by the method 170. As demonstrated in the preliminary results captured, some of which were previously discussed, the bioimpedance data associated with the assessment routine 90 may provide for reliable indicators that suggest both the relative severity and, in some cases, the type or category of the injury 14. However, the specific impedance values measured with the routine 90 may differ based on a number of variables. Such variations in the impedance results may be limited by examining the percent change in impedance (e.g., resistance and/or reactance) across symmetric segments 20a, 20b of the body. Similarly, the assessment of the severity and the recovery rate of an injury for the patient 12 may be accurately tracked over time based on a series of measurements of the impedance for a single segment 20 compared to a baseline for the segment 20 in a healthy condition or detected over several milestones following an injury. The assessment routine 170 may apply the subject data, injury or condition data, historic impedance data (step 182) from the routine 90, and/or additional sensor data (step 184) to identify trends in impedance data and improve the accuracy of the preliminary results with a trained assessment model as applied in step 186.

The trained assessment model may be trained based on a library of ground truth or confirmed diagnostic results associated with the diagnoses of clinicians based on CT, X-ray, and/or MRI data captured associated with injuries (e.g., muscle injuries, tendon injuries, various grades of soft tissue inflammation, etc.) that also have measured results captured with the impedance assessment routine 90. Inputs to the training procedure for the method may also include inputs associated with one or more of the subject data, injury or condition data, historic impedance data (step 182) from the routine 90, and/or additional sensor data (step 184). Accordingly, the trained model may be supplied with various datapoints that identify the nature of the patient, the conditions of the injury, as well as bioelectrical impedance and/or additional sensor data that may assist in the determination of the type or category of the injury 14 reported by the patient 12. Alternatively, the assessment method 170 may similarly be implemented to refute the denial of the injury 14 as reported by an athlete or employee who may be otherwise motivated to avoid an activity restriction. In this way, the assessment method 170 may provide for preliminary indications that may be effectively applied to suggest the best follow-up course for the patient 12.

As discussed in reference to step 182, the historic assessment data from the assessment routine 90 may be supplied to the assessment model applied in step 186. The historic assessment data may be informative to demonstrate a rate of change of the results from the assessment routine 90, which may be applied to infer the type of the injury 14 or condition of the patient 12. Similarly, the historic assessment data may provide information regarding the association between various metrics from the subject data and/or the patient data to inform the model how the patients studied are able to return to activity or how they subjectively feel over a recovery period. While such inputs may be less directly related to the resulting preliminary classification or identification of the type of the injury, this information may assist in grouping similar types of injuries in relation to the temporal progression of recovery periods for a variety of injuries.

The additional sensor data captured in step 184 may provide additional indicators that may be associated with the severity or type of condition or injury 14 reported by the patient 12. For example, the heart rate variability (HRV) may serve as an indicator of injury or a recovery level of the patient 12. Similarly, blood oxygen levels as well as hydration levels associated with the patients may correlate to variations in the bioelectrical impedance measurements captured with the routine 90. Accordingly, the additional sensor data captured in step 184 may provide inputs to the assessment model that may normalize the variations among the bioelectrical impedance measurements among patients to further improve the accuracy of the preliminary assessment into the type, classification, and/or severity of the injury. Accordingly, the disclosure may provide for preliminary assessments in relation to a variety of injuries or physiological conditions.

Still referring to FIG. 12, the assessment model of step 186 may output the potential conditions, an injury type, a severity, and/or a classification of the injury 14. Based on the preliminary determination of the assessment, follow up steps may also be suggested including additional scanning, appointments with medical professionals, activity restrictions, and may even include one or more proposed treatment plans that may be supervised by qualified providers to ensure the assessment method 170 is properly implemented. Such assessment information may be provided in the form of a report to a remote homecare, telepresence medical professional, an individual, private organizations, etc. to provide meaningful insight as to the condition and health of the patient 12 to assist in a comprehensive treatment plan. Finally, the method 170 may record and report the assessment data to a database that may be implemented to improve the accuracy of the assessment model by tracking the outcomes of the associated patients 12 that utilize the method 170 through ongoing treatment. In this way, the operation of the assessment method 170 may continue to improve and continue to tune the nodes and relationships among the inputs to improve with accuracy over time.

Referring now to FIG. 13, the disclosed methods, diagnostic routines, and monitoring equipment may be implemented as part of a connected monitoring system 200. In the example shown, the monitoring system 200 may include various pieces of monitoring equipment 202 that may be in communication via a network of devices. In various implementations, the monitoring equipment 202 may be in communication with various interface devices 204 that may include mobile phones, tablets, computers, etc. that may be utilized by one or more users, subjects, and/or administrators to enter subject data and access monitoring/evaluation results as discussed herein. In this way, various users and administrators of the monitoring equipment 202 may access and interact with the monitoring equipment 202 via a variety of interface devices to enter information and/or review results.

In various implementations, the monitoring equipment 202 may be configured to record subject data and/or user profiles 206 to a secure, profile database 208. In this configuration, the subject data and/or user profiles 206 may be accessed by the monitoring equipment 202 within the subject database to access historic test data. As previous disclosed, the historic data may include baseline information and/or variations in bioimpedance over time for each of the subjects that utilize the monitoring equipment 202. In some implementations, the subject data may be securely accessed by one or more authorized medical professionals who may review the monitoring data associated with the bioimpedance analysis to assist in identifying various stages of pre-injury, injury, and recovery for patients who may be monitored as part of a comprehensive monitoring routine. Additionally, the medical professional accessing the diagnostic portal may add corresponding clinical diagnostic information associated with various clinical evaluations or testing (e.g., MRI, CT, etc.). In this way, medical diagnostic data may be reported via a clinician portal 210. Accordingly, the clinician portal 210 may include medical diagnostic information that may be utilized to validate the corresponding bioimpedance results captured by the monitoring equipment 202. The diagnostic input provided by the physicians or authorized medical professionals may serve to improve one or more resulting trained, predictive models 212 as later discussed.

The subject data and/or user profiles 206 may be processed through a filtering step 214 that may remove and/or purge any personal data associated with the corresponding profiles 206. Once filtered, the corresponding bioimpedance and/or diagnostic data entered through the portal 210 may be uploaded to an anonymous user database 216. The anonymous user database may serve as a repository for training data 218 that may be accessed to train one or more predictive models via various model-training procedures exemplified by the model-training block 220. For example, the training data 218 entered into the model-training block 220 may serve to generate a wide variety of predictive and/or analytical models that may be utilized to process the bioimpedance data to provide pre-diagnostic indications for a wide variety of potential injuries and/or provide training and/or risk assessment feedback to users. The access of the aggregate data from the user database 216 may allow the resulting predictive or trained model 212 to account for a wide variety of variations in testing equipment, preexisting conditions, and/or variations in the reported bioimpedance data to further improve the accuracy of the corresponding indications in reference to the identification of one or more potential injuries, risk conditions, and/or recovery/training statuses for the various users of the system 200.

Finally, the routine data captured from the impedance analysis with the monitoring equipment 202 may be automatically processed via the analytical or predictive model 212. For example, a subject having completed an evaluation or measurement with the monitoring equipment 202 may receive automated results providing indications of various health criteria including, but not limited to, various stages of pre-injury, strain, injury, and recovery. The results may be presented to the subject/user via one of the interface devices 204. In this way, each of the subjects may receive status updates of the various indicators of the associated tissue health, which may serve as guidance to adjust exercise routines and/or seek medical diagnosis or treatment. Further, in a supervised routine, health professionals may oversee the training, recovery, and/or potential injuries of patients and receive automated alerts identifying increased risk or injury indications via the clinician portal 210.

As previously discussed, the monitoring equipment 202 may include a variety of sensors that may capture additional sensor data that may be indicative of the state of recovery, severity, type of condition or injury, etc. The heart rate variability (HRV) may serve as an indicator of injury or of a recovery level of the patient 12. Similarly, blood oxygen levels as well as hydration levels associated with the patients may correlate to variations in the bioelectrical impedance measurements captured with the routine 90. In various cases, the additional sensor data may provide additional inputs to the training data 218 that may improve and/or validate the model training 220. In this way, the predictive model or assessment model 212 may normalize the variations among the bioelectrical impedance measurements among patients to further improve the accuracy of the preliminary assessment into the type, classification, and/or severity of the injury or stage of recovery. Accordingly, the disclosure may provide for preliminary assessments in relation to a variety of injuries or conditions.

According to some aspects of the disclosure, an assessment method for identifying tissue abnormalities and injuries comprises supplying an excitation signal via a plurality of source electrodes into a plurality of anatomical segments of a patient, wherein the anatomical segments are on opposing side of the body of the patient; detecting characteristic signals resulting from a first impedance of a first segment and a second impedance of a second segment of the anatomical segments; determining a difference between the first impedance compared to the second impedance; and attributing an injury to either the first segment or the second segment in response to the difference exceeding a first threshold.

According to various aspects, the disclosure may implement one or more of the following features or configurations in various combinations:
compare the difference to a second threshold;
in response to the difference exceeding the second threshold, identify a relative severity of the injury or tissue condition;
the relative severity of the injury or tissue condition may indicate that an advanced medical intervention (e.g., specialist referral, rehabilitation, scanning, MRI, etc.) is necessary to treat the injury or tissue condition;
identify the injury or tissue condition in the first segment in response to the first impedance exceeding the second impedance;
identify the injury or tissue condition in the second segment in response to the second impedance exceeding the first impedance;
the impedance comprises at least one of a resistance and a reactance identified in response to changes between the first impedance and the second impedance;
conductively connecting the plurality of source electrodes and sense electrode across the first segment and the second segment; and/or
the first segment and the second segment are bilaterally symmetric across a sagittal plane of the patient.

It will become apparent to those skilled in the art that various modifications to the preferred embodiment of the invention as described herein can be made without departing from the spirit or scope of the invention as defined by the appended claims.

The invention claimed is:

1. An assessment method for identifying tissue conditions and injuries comprising:
supplying an excitation signal via a plurality of source electrodes into a plurality of anatomical segments of a patient, wherein the anatomical segments are on opposing side of the body of the patient;
detecting characteristic signals resulting from a first impedance of a first segment and a second impedance of a second segment of the anatomical segments;
determining a difference between the first impedance compared to the second impedance; and
attributing an injury or tissue condition to either the first segment or the second segment in response to the difference exceeding a first threshold.

2. The assessment method according to claim 1, further comprising:
comparing the difference to a second threshold; and
in response to the difference exceeding the second threshold, identifying a severity of the injury.

3. The assessment method according to claim 1, wherein the severity indicates if medical intervention is necessary to manage the tissue condition.

4. The assessment method according to claim 1, wherein attributing the injury comprises:
identifying the injury in the first segment in response to the first impedance exceeding the second impedance; and
identifying the injury in the second segment in response to the second impedance exceeding the first impedance.

5. The assessment method according to claim 1, wherein the impedance comprises at least one of a resistance and a reactance identified in response to the characteristic signals.

6. The assessment method according to claim 5, wherein the difference is calculated as a percent difference of the reactance of the first impedance and the second impedance.

7. The assessment method according to claim 1, further comprising:
conductively connecting the plurality of source electrodes and sense electrode across the first segment and the second segment.

8. The assessment method according to claim 1, wherein the first segment and the second segment are bilaterally symmetric across a sagittal plane of the patient.

9. A health assessment device configured to detect a tissue health of a subject, the assessment device comprising:
at least one controller in communication with a signal generator configured in communication with a plurality of source electrodes configured to conductively connect to a first portion of the subject, where the controller is further in communication with a plurality of sense electrodes configured to conductively connect to a second portion of the subject across at least one body segment from the first portion, wherein the controller is configured to:
activate an excitation signal output from the signal generator to the source electrodes;
detect at least one characteristic signal resulting from the excitation signal conducted through the at least one body segment; and
identify an impedance difference between a first impedance of a first segment of the at least one segment relative a baseline impedance of the first segment or concurrent a second impedance of a second segment symmetrical to the first segment, wherein the impedance difference is indicative of a diminished tissue condition of the first segment relative to the baseline impedance or the second segment.

10. The health assessment device according to claim 9, wherein in response to the impedance difference exceeding a first percent difference, the diminished tissue condition is identified to correspond to a minor injury category.

11. The health assessment device according to claim 9, wherein the controller is further configured to:
receive subject data identifying at least one of an age, a pre-existing condition, and a demographic of the subject.

12. The health assessment device according to claim 11, wherein the controller is further configured to:
receive injury or tissue condition data identifying at least one of an injury timing (e.g., how recent), an activity type, a segment/body location, a perceived severity (e.g., swelling, range of motion, etc.), a pain level, and an acute versus chronic indication associated with the diminished tissue condition.

13. The health assessment device according to claim 12, wherein the controller is further configured to:
identify a potential condition with a trained model in response to the impedance difference in combination with at least one of the subject data and the condition data.

14. The health assessment device according to claim 11, wherein the controller is further configured to: identify at least one of a list, a type, a severity, and a category of potential conditions or injuries with the trained model.

15. The health assessment device according to claim 11, wherein the excitation signal is between 1 kHz and 250 kHz, or wherein the excitation signal is between 40 kHz and 60 kHz.

16. The health assessment device according to claim 9, wherein the impedance difference comprises at least one of a resistance and a reactance identified in response to the characteristic signals.

17. The health assessment device according to claim 9, wherein the at least one body segment comprises a plurality of segments including the first segment symmetrically positioned across a sagittal plane of the subject from the second segment.

18. A system for soft tissue monitoring comprising:
at least one bioimpedance testing device configured to identify bioimpedance data for a plurality of subjects;
a profile database comprising a plurality of user profiles to which the corresponding bioimpedance data for the plurality of subjects assigned and stored, the profile database in communication with the at least one bioimpedance device;
at least one controller in communication with the at least one bioimpedance testing device and the profile database, wherein the controller is configured to:
receive the bioimpedance data from the plurality of subjects;
process the bioimpedance data via an analytical model; and
in response to the processing of the bioimpedance data, generates an assessment of a tissue condition of the associated subject based on the analytical model.

19. The system according to claim 18, wherein the analytical model processes the bioimpedance data for an associated subject by comparing the bioimpedance data to a historic bioimpedance measurement of the associated subject.

20. The system according to claim 19, wherein the comparison of the bioimpedance data to a historic bioimpedance measurement of the associated subject comprises comparing the bioimpedance data for at least one segment anatomical segment of the associated subject to an average or baseline of the historic bioimpedance measure of the associated subject.

21. The system according to claim 18, wherein the analytical model processes the bioimpedance data for an associated subject by comparing a difference between bilaterally offset anatomical segments identified in the bioimpedance data.

22. The system according to claim 18, wherein the analytical model processes the bioimpedance data for an associated subject by comparing the bioimpedance data to anonymous composite data associated with the plurality of subjects.

23. The system according to claim 18, wherein the analytical model is generated from anonymous data processed from the bioimpedance data stored for the plurality of subjects via a model training process.

24. The system according to claim 23, wherein the bioimpedance data for at least a portion of subjects is validated by one or more qualified users as validated data identifying a correspondence of the bioimpedance data to one or more diagnosed conditions.

25. The system according to claim 24, wherein the validated data is provided as an input to the model training process and identifies the correspondence of the bioimpedance data to one or more diagnosed conditions.

26. The system according to claim 24, wherein the one or more diagnosed conditions corresponding to a health condition of a joint or tissue.

27. The system according to claim 26, wherein the health condition comprises at least one of a ligament condition, a muscle condition, or a tendon condition indicating a state of injury or recovery.

28. The system according to claim 24, further comprising:
a user portal comprising a user interface providing access to qualified users to access the bioimpedance data for one or more of the plurality of subjects, wherein the user portal is accessed by the qualified users to validate the bioimpedance data in correspondence with the diagnosed condition.

29. The system according to claim 18, wherein the bioimpedance data for one or more of the plurality of subjects is captured and evaluated with the at least one bioimpedance testing device periodically in an ongoing monitoring routine.

30. The system according to claim 18, wherein the at least one bioimpedance testing device comprises a plurality of bioimpedance devices in communication with the profile database.
